(19) United States
(12) Patent Application Publication
(10) Pub. No.: US 2010/0324374 A1
(43) Pub. Date: Dec. 23, 2010

(54) LARYNGEAL STROBOSCOPE USING VOICE SIGNAL

(75) Inventors: Byoung Chul Kim, Gyeongsangbuk-Do (KR); Kyong Hae Park, Busan (KR); Ki Sik Kown, Busan (KR); Bum Yong Choi, Seoul (KR); Jong Won Park, Busan (KR); Kyu Won Lee, Busan (KR); Jin Choon Lee, Busan (KR)

Correspondence Address: PEARNE & GORDON LLP, 1801 EAST 9TH STREET, SUITE 1200, CLEVELAND, OH, 44114-3108, US

(73) Assignee: UMEDICAL CO., LTD., Busan (KR)

(21) Appl. No.: 12/666,162

(22) PCT Filed: May 29, 2008
(86) PCT No.: PCT/KR08/03004

§ 371 Date: Dec. 22, 2009

(30) Foreign Application Priority Data
Jul. 9, 2007 (KR) 20-2007-0011272

(51) Int. Cl. A61B 1/267

(52) U.S. Cl. 600/199

(57) ABSTRACT

Provided is a laryngeal stroboscope using voice signals. The laryngeal stroboscope includes a photography unit for photographing a patient's vocal cords and a light source for illuminating a part to be photographed, a microphone for sensing the patient's voice, a high-pass filter for passing voice signals of 100 Hz or more among voice signals output from the microphone, a low-pass filter for removing signals of harmonic components from the voice signals output from the high-pass filter and passing only a voice signal in a fundamental frequency band, a peak detector for detecting a peak of the voice signal output from the low-pass filter and obtaining a pitch, and a controller for controlling the light source to intermittently emit light in response to a signal output from the peak detector and controlling the photography unit to operate according to whether or not the light is intermittently emitted.

### TECHNICAL FIELD

The present invention relates to a laryngeal stroboscope for photographing vocal cords in connection with an endoscope, and more particularly, to a laryngeal stroboscope synchronizing light emission with a voice signal using a microphone.

### BACKGROUND ART

A stroboscope is an instrument that radiates cyclically-blinking light to a rapidly-rotating or vibrating object and thereby enables the object to be observed as though it were stationary or slow-moving. A laryngeal stroboscope synchronizes light emission operations with the fundamental frequency of vocal cords, thereby enabling a user to observe the vocal cords that continuously vibrate during utterance but appear to be stationary at one phase point or slow-moving. Such a laryngeal stroboscope is a useful instrument for examining minute lesions of vocal cords, the prognosis of vocal cord paralysis, and so on.

In fact, a laryngeal stroboscope does not show an image every cycle but shows a composite image obtained from images of many continuous laryngeal cycles. Therefore, a laryngeal stroboscope basically synchronizes a stroboscope signal with the fundamental frequency of vocal cords. However, a process of synchronizing the stroboscope signal with the fundamental frequency of vocal cords in a laryngeal stroboscopic examination is very complicated. In addition, when the synchronization is not perfect, vibration of the vocal cords cannot be correctly observed.

In other words, the most difficult problem in developing a laryngeal stroboscope is to find a method of accurately detecting the fundamental frequency, i.e., the pitch, of an examinee's vocal cords, and a method of generating a peak having an apparent difference in one cycle.

Thus far, the following pitch detection methods have been developed. A first method uses an Electroglottograph (EGG), which is used by most laryngeal stroboscopes currently put on the market. An EGG always shows a waveform having one peak in one frequency while accurately detecting a glottal closure instant using a differentiated EGG, and thus is preferred over other methods. However, an EGG device is additionally required, and electrodes are placed on either side of an examinee's thyroid cartilage plate at each examination. In addition, the EGG device has a large volume and thus is difficult to move. Furthermore, the EGG device is very expensive and cannot be used in most hospitals except university hospitals.

A second method uses a vibration pick-up. A patient's voice signal is sensed as vibration and synchronized with light emission after specific signal processing. As the vibration pick-up, a vibration sensor, etc., may be used. When vibration of vocal cords and a change in airway pressure are measured by a sensor for vibration measurement, the pitch of a fundamental frequency corresponding to vocal fold vibration can be directly extracted, unlike voice passing through a vocal organ and having various high frequencies.

According to a third method, a pitch is detected by a stethoscope that is connected with a microphone and put on a subglottic thyroid cartilage plate. All laryngeal stroboscopes developed in the 1980's use this method, but nowadays its use is declining. This is because the third method is a bit less accurate in pitch detection than the first method using an EGG device, and it is inconvenient to put the stethoscope on a patient's neck.

The peaks of voice signals of most male and female patients are distributed within a band of 120 Hz to 900 Hz, and the frequency of a peak point rapidly varies. Thus, when a microphone is used, it is difficult to detect the peak of a fundamental wave signal, and there is a problem in the speed of an Automatic Gain Control (AGC) operation directly related to peak signal detection. Consequently, the light emission cycle of a stroboscope cannot be accurately determined with respect to most voice samples.

### DISCLOSURE

### Technical Problem

The present invention provides a laryngeal stroboscope that removes a peak signal of harmonic components excluding a fundamental frequency of a voice signal, to prevent irregular movement of a peak point and enable perfect synchronization with a strobe light.

### Technical Solution

The present invention discloses a laryngeal stroboscope, including: a laryngeal endoscope including a photography unit for photographing a patient's vocal cords and a light source for illuminating a part to be photographed; a microphone for sensing the patient's voice; a high-pass filter for passing voice signals of 100 Hz or more among voice signals output from the microphone; a low-pass filter for removing signals of harmonic components from the voice signals output from the high-pass filter, and passing only a voice signal in a fundamental frequency band; a peak detector for detecting a peak of the voice signal output from the low-pass filter and obtaining a pitch; and a controller for controlling the light source to intermittently emit light in response to a signal output from the peak detector, and controlling the photography unit to operate according to whether or not the light is intermittently emitted.

The microphone may not be put on the patient's body.

The low-pass filter may pass a signal of 350 Hz or less. The laryngeal stroboscope may further include an amplifier for amplifying the voice signal output from the low-pass filter and transferring the amplified voice signal to the peak detector.

The laryngeal stroboscope may further include a display for displaying a laryngeal image taken by the photography unit. The light source may use a Light-Emitting Diode (LED). The photography unit may be a Charge-Coupled Device (CCD) camera.

### ADVANTAGEOUS EFFECTS

As is apparent from the above description, the laryngeal stroboscope according to an exemplary embodiment of the present invention passes a low-band signal among voice signals of an examinee using a microphone attached thereto, without putting any mechanical instrument on the patient's neck, and thus is perfectly synchronized with strobe light.

### DESCRIPTION OF DRAWINGS

The accompanying drawings, which are included to provide a further understanding of the invention and are incorporated in and constitute a part of this specification, illustrate exemplary embodiments of the invention, and together with the description serve to explain the aspects of the invention.

FIG. 1 is a block diagram of a laryngeal stroboscope according to an exemplary embodiment of the present invention.

FIG. 2 is a circuit diagram of a low-pass filter shown in FIG. 1 according to an exemplary embodiment of the present invention.

FIGS. 3, 5 and 7 illustrate input spectrums of patients' voice signals input to a laryngeal stroboscope according to an exemplary embodiment of the present invention, and FIGS. 4, 6 and 8 illustrate output spectrums of the patients' voice signals output from the laryngeal stroboscope according to the exemplary embodiment of the present invention.

FIG. 9 illustrates a change between input and output signals of a laryngeal stroboscope according to an exemplary embodiment of the present invention.

### MODE FOR INVENTION

The invention is described more fully hereinafter with reference to the accompanying drawings, in which exemplary embodiments of the invention are shown. This invention may, however, be embodied in many different forms and should not be construed as limited to the exemplary embodiments set forth herein. Rather, these exemplary embodiments are provided so that this disclosure is thorough, and will fully convey the scope of the invention to those skilled in the art. Like reference numerals in the drawings denote like elements.

### EXEMPLARY EMBODIMENT

An exemplary embodiment of the present invention will be described below in detail with reference to accompanying drawings.

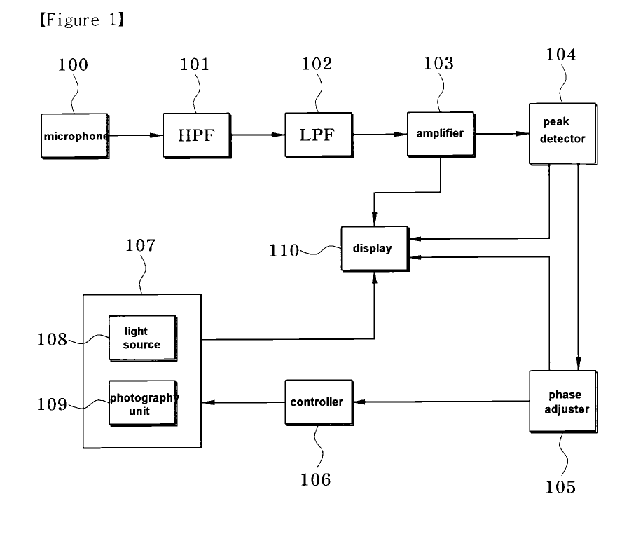

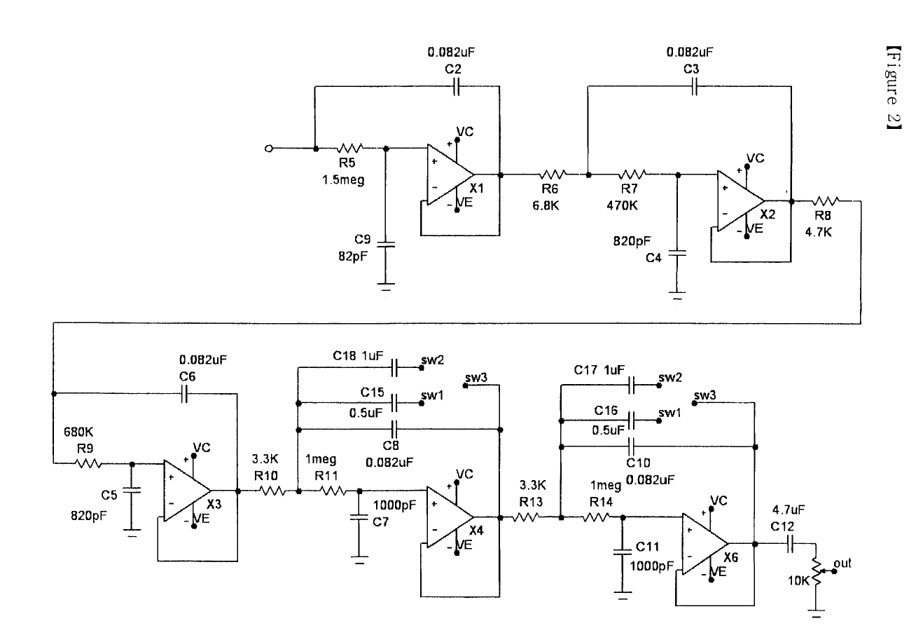

FIG. 1 is a block diagram of a laryngeal stroboscope according to an exemplary embodiment of the present invention. Referring to FIG. 1, the laryngeal stroboscope according to an exemplary embodiment of the present invention comprises a microphone 100, a high-pass filter 101, a low-pass filter 102, an amplifier 103, a peak detector 104, a display 110, a phase adjuster 105, a controller 106, and a laryngeal endoscope 107.

The microphone 100 serves to sense a patient's voice signal. Unlike conventional art, the microphone 100 is not put on the patient's neck, etc. A voice signal output from the microphone 100 passes through the high-pass filter 101 and the low-pass filter 102.

The high-pass filter 101 passes only signals of a high frequency band among voice signals. In this exemplary embodiment, the high-pass filter 101 is used to obtain voice signals of 100 Hz or more because voice signals of less than 100 Hz are meaningless, as described below.

The low-pass filter 102 passes only signals of a low frequency band among voice signals. In this exemplary embodiment, the low-pass filter 102 may pass signals of 350 Hz or less. The peak points of men's voice signals are irregularly distributed within a band of 120 Hz to 600 Hz, and that of women's voice signals are irregularly distributed within a band of 250 Hz to 900 Hz. Thus, the peak points of most male and female patients' voices are irregularly distributed within a band of 120 Hz to 900 Hz. Since the frequency of a peak point rapidly varies, the low-pass filter 102 is used to remove harmonic components of a peak signal leaving only a fundamental frequency.

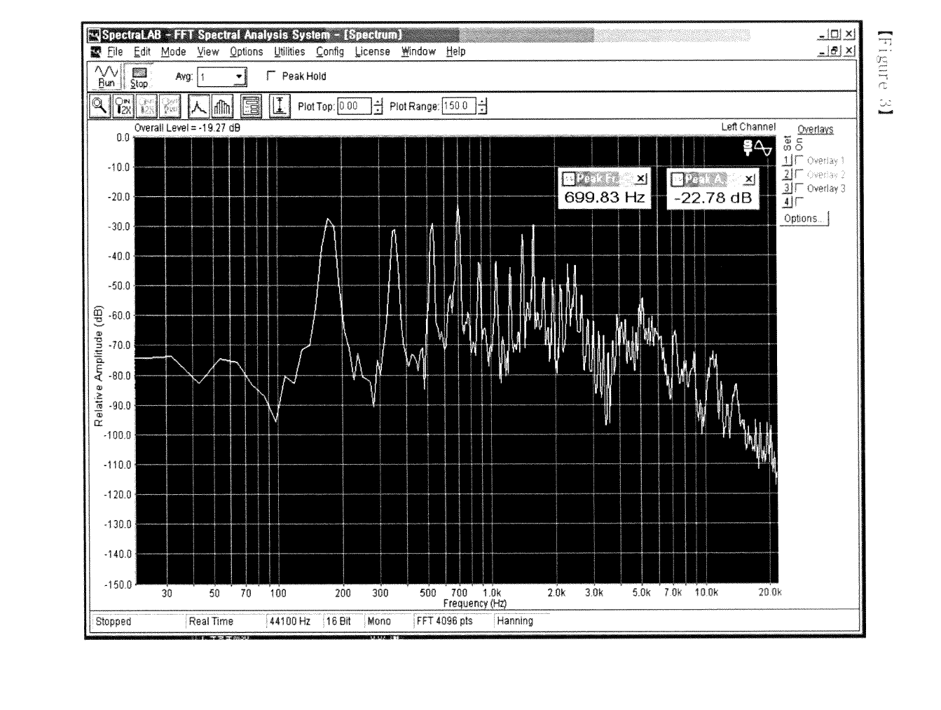

FIG. 2 is a circuit diagram of the low-pass filter shown in FIG. 1, which is a fifth-order active filter designed using an Operational Amplifier (OP-AMP).

The amplifier 103 serves to amplify a low-passed voice signal with a specific ratio or more and facilitate peak detection.

The peak detector 104 serves to detect one peak in one cycle of a voice waveform and obtain the pitch of a voice signal. This is because an accurate pitch must be detected from a voice signal collected from the microphone to synchronize a light source of the stroboscope with the voice signal. The phase adjuster 105 serves to adjust the phase of the signal whose peak is detected.

The controller 106 controls a light source 108 to intermittently emit light in response to a signal input from the phase adjuster 105, and a photography unit 109 to photograph a patient's larynx according to whether or not the light is intermittently emitted. Therefore, the light source 108 is repeatedly turned on and off in synchronization with the frequency of the patient's voice signal.

The laryngeal endoscope 107 is intended to observe the state of a larynx, and may use any well-known laryngeal endoscope. As illustrated in FIG. 1, the laryngeal endoscope 107 includes the photography unit 109 and the light source 108. The photography unit 109 is intended to photograph the state of a larynx, i.e., vocal cords, and may use a general small Charge-Coupled Device (CCD) camera. Here, a lens of the photography unit 109 may be disposed at the front end of the endoscope 107, and the CCD camera, a driver circuit, etc., may be disposed at the rear part of the endoscope 107. The light source 108 is installed adjacent to the lens of the photography unit 109 and radiates light to vocal cords, thereby serving as lighting. The light source 108 may use a Light-Emitting Diode (LED) to reduce power consumption, production cost and weight. In particular, a white LED may be preferred. The light source 108 may include a plurality of LEDs to generate a luminance that a user wants.

Images taken in this way are transferred to the display 110 so that a doctor can observe them. The display 110 may be any display means such as a Liquid Crystal Display (LCD).

The above-described laryngeal stroboscope according to an exemplary embodiment of the present invention includes an endoscope, but another exemplary embodiment may have the same constitution except the endoscope. When an endoscope is not included, a laryngeal stroboscope according to an exemplary embodiment of the present invention may be used in connection with a well-known endoscope having a light source and a photography unit. In addition, a laryngeal stroboscope according to an exemplary embodiment of the present invention may be wired and use a general power supply, or use a battery and a wireless transceiver to be carried along.

An experimental embodiment of the present invention will be described below.

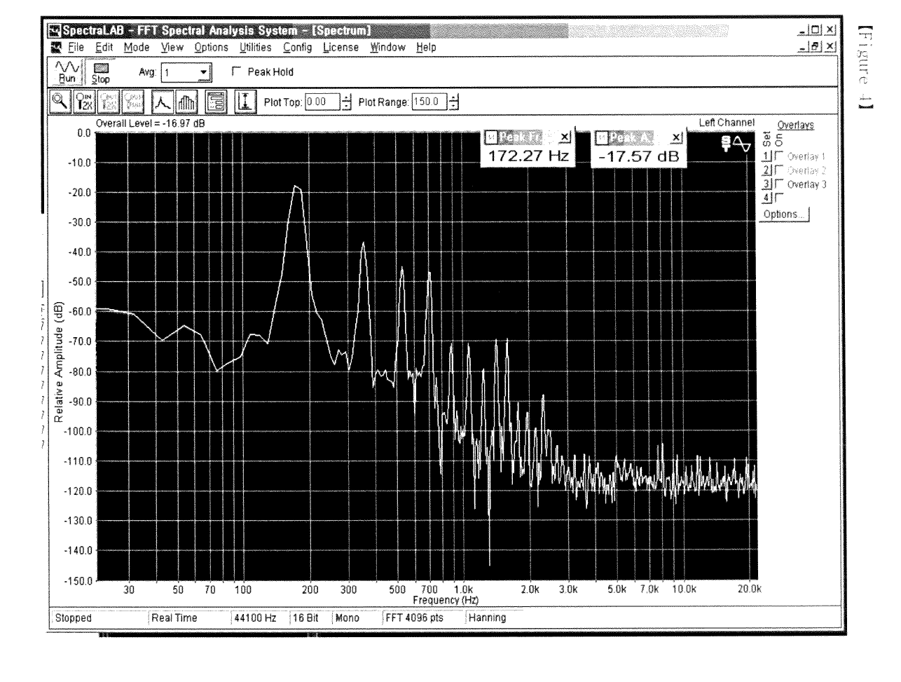

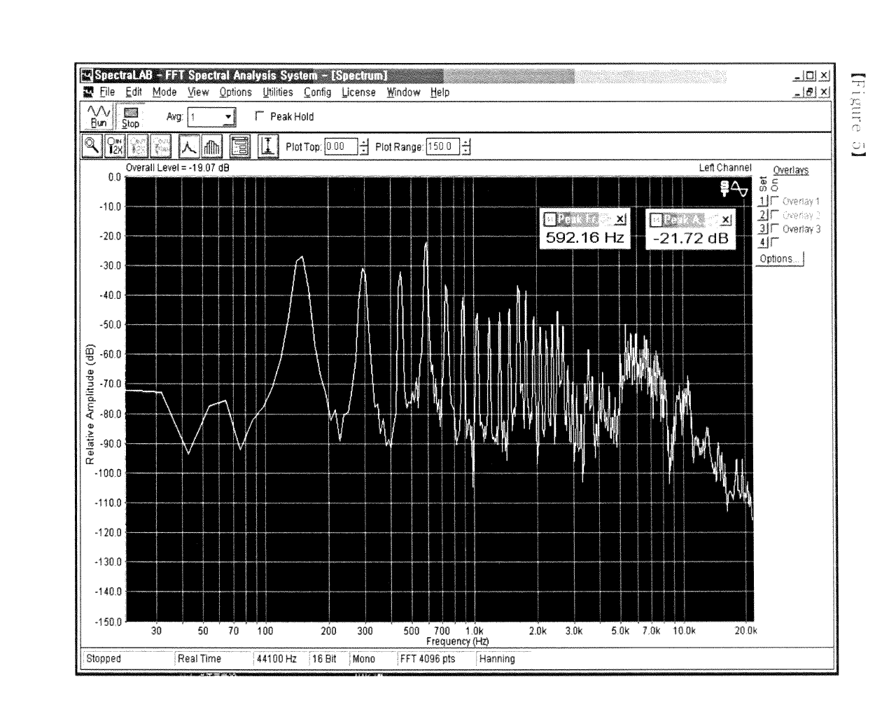

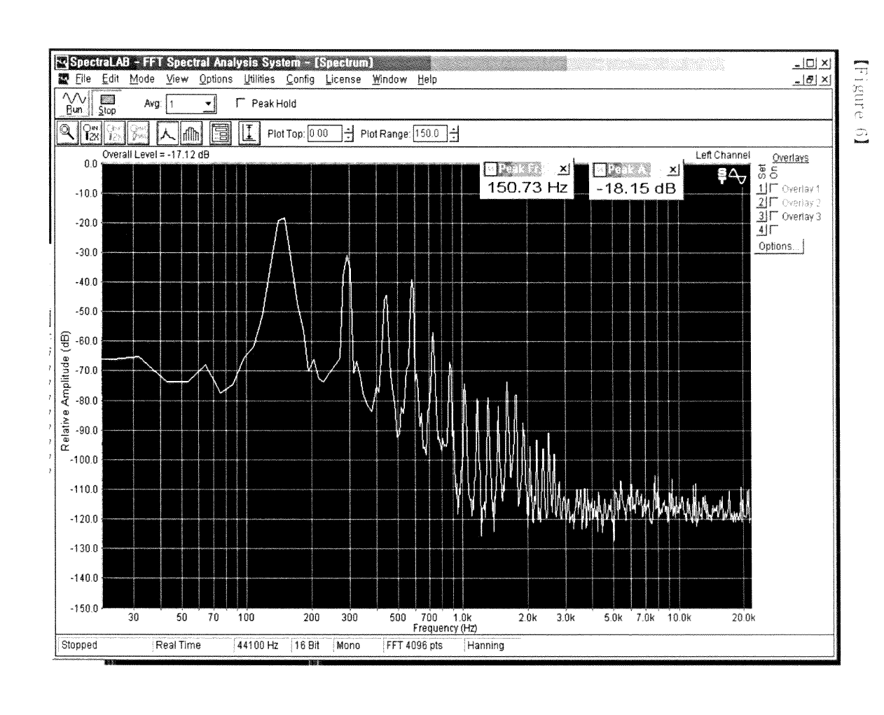

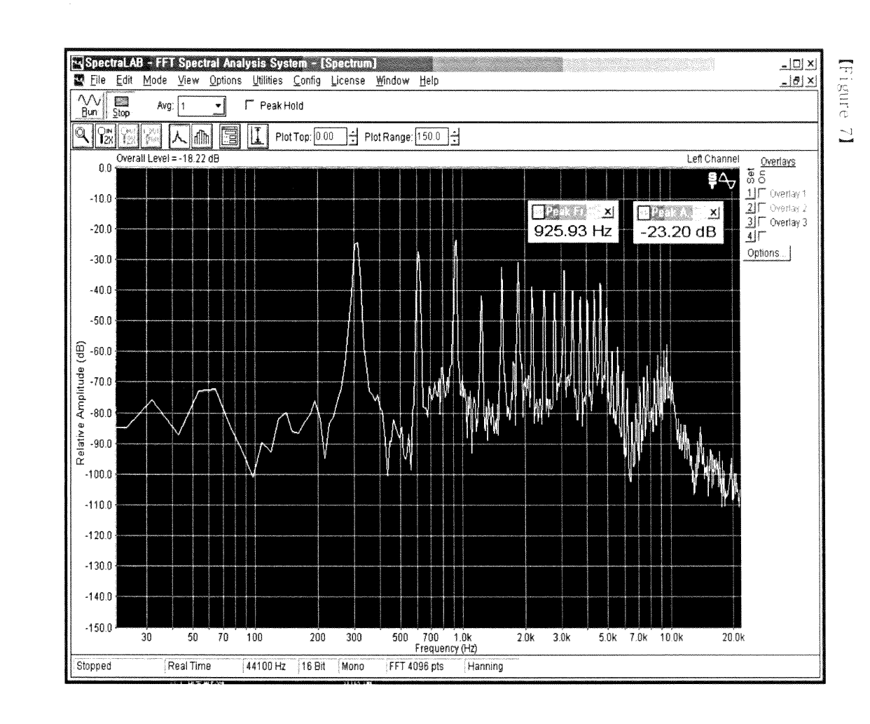

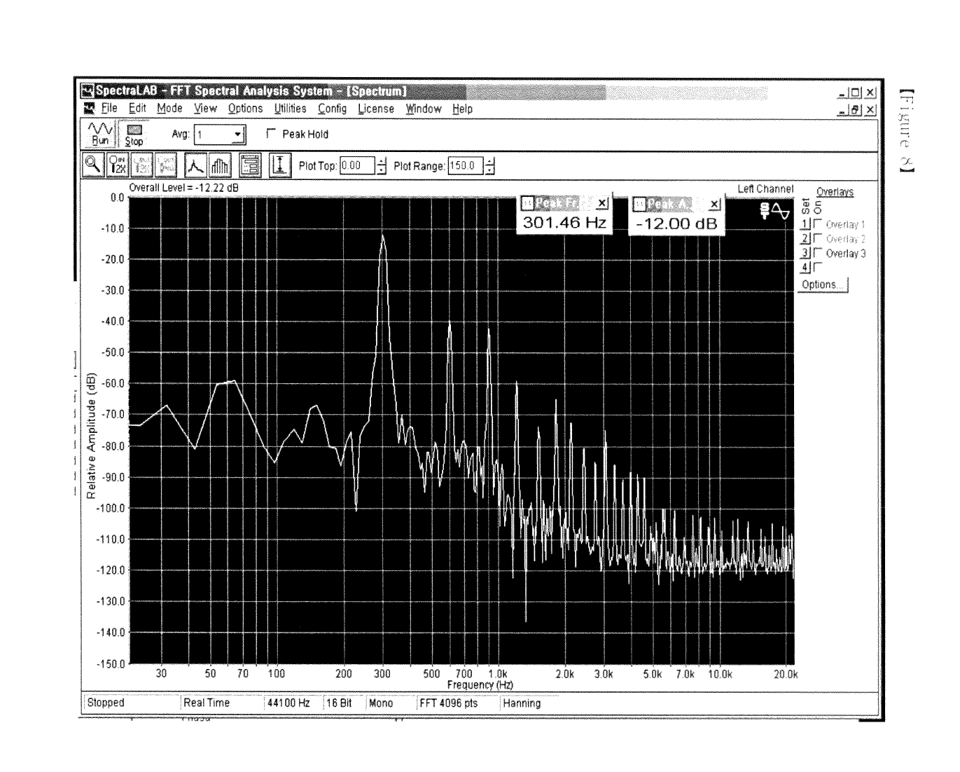

FIGS. 3, 5 and 7 illustrate input spectrums of patients' voice signals input to a laryngeal stroboscope according to an exemplary embodiment of the present invention, and FIGS. 4, 6 and 8 illustrate output spectrums of the patients' voice signals output from the laryngeal stroboscope according to the exemplary embodiment of the present invention. Referring to FIGS. 3, 5 and 7, peak frequencies of the input spectrums of the patients' voice signals are 699.83 Hz, 592.16 Hz and 925.93 Hz. Referring to FIGS. 4, 6 and 8, peak frequencies of the output spectrums of the patients' voice signals output from a laryngeal stroboscope according to an exemplary embodiment of the present invention through a high-pass filter and a low-pass filter are changed into 172.27 Hz, 150.73 Hz and 301.46 Hz.

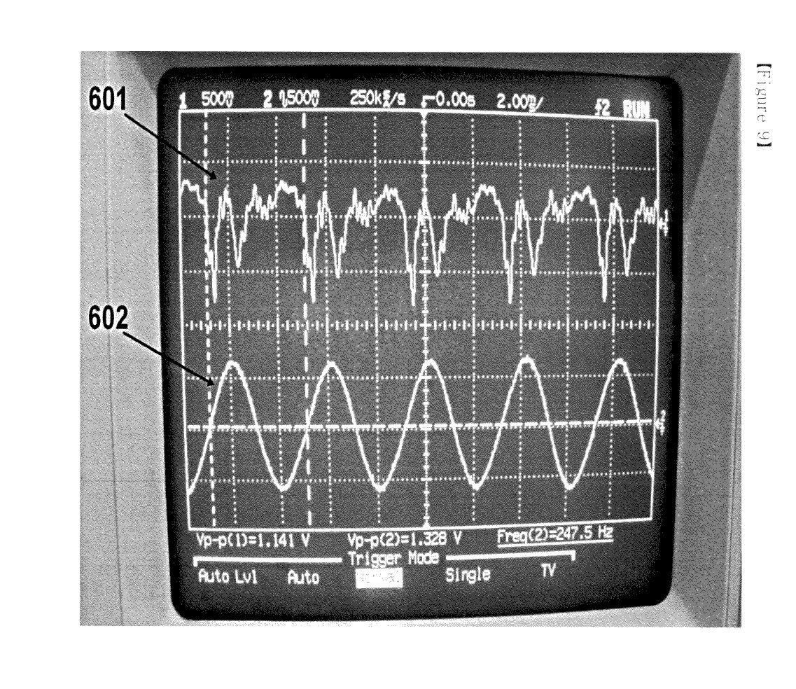

FIG. 9 illustrates a change between input and output signals of a laryngeal stroboscope according to an exemplary embodiment of the present invention. In FIG. 9, a graph 601 indicates an input voice signal, and a graph 602 indicates a voice signal output from a laryngeal stroboscope according to an exemplary embodiment of the present invention. It can be seen that peaks are more apparent in the output signal than in the input signal.

It will be apparent to those skilled in the art that various modifications and variations can be made in the present invention without departing from the spirit or scope of the invention. Thus, it is intended that the present invention covers the modifications and variations of this invention provided they come within the scope of the appended claims and their equivalents.

1. A laryngeal stroboscope, comprising:
a laryngeal endoscope including a photography unit for photographing a patient's vocal cords and a light source for illuminating a part to be photographed;
a microphone for sensing the patient's voice;
a high-pass filter for passing voice signals of 100 Hz or more among voice signals output from the microphone;
a low-pass filter for removing signals of harmonic components from the voice signals output from the high-pass filter, and passing only a voice signal in a fundamental frequency band;
a peak detector for detecting a peak of the voice signal output from the low-pass filter and obtaining a pitch; and
a controller for controlling the light source to intermittently emit light in response to a signal output from the peak detector, and controlling the photography unit to operate according to whether or not the light is intermittently emitted.

2. The laryngeal stroboscope of claim 1, wherein the microphone is not put on the patient's body.

3. The laryngeal stroboscope of claim 1, wherein the low-pass filter passes a signal of 350 Hz or less.

4. The laryngeal stroboscope of claim 1, further comprising:
an amplifier for amplifying the voice signal output from the low-pass filter and transferring the amplified voice signal to the peak detector.

5. The laryngeal stroboscope of claim 1, further comprising:
a display for displaying a laryngeal image taken by the photography unit.

6. The laryngeal stroboscope of claim 1, wherein the light source uses a Light-Emitting Diode (LED).

7. The laryngeal stroboscope of claim 1, wherein the photography unit is a Charge-Coupled Device (CCD) camera.
